(19) United States
(12) Patent Application Publication
(10) Pub. No.: US 2006/0121591 A1
(43) Pub. Date: Jun. 8, 2006

(54) Mutant proteinase-inhibitors and uses thereof

(75) Inventors: Daniel A. Lawrence, Derwood, MD (US); Natalia Gorlatova, Rockville, MD (US); David L. Crandall, Doylestown, PA (US)

Correspondence Address: FOLEY AND LARDNER LLP;SUITE 500, 3000 K STREET NW, WASHINGTON, DC, 20007, US

(73) Assignees: American National Red Cross; WYETH

(21) Appl. No.: 11/341,567

(22) Filed: Jan. 30, 2006

Related U.S. Application Data
(62) Division of application No. 10/197,258, filed on Jul. 18, 2002.
(60) Provisional application No. 60/305,908.

(51) Int. Cl. C12N 9/99

(52) U.S. Cl. 435/184

(57) ABSTRACT

A library of mutants of metastable proteins, such as proteinase inhibitors, can be screened for the specific loss of a wild-type capability to bind an antibody, yielding valuable drug-design information which otherwise is unavailable. By this approach, for example, a mutant proteinase inhibitor can be obtained that has the amino acid sequence of a wild-type protein, or an active fragment thereof, save for the presence of one or more mutations in at least one epitope, thereby altering interaction of the mutant with an anti-proteinase inhibitor antibody.

### BACKGROUND OF THE INVENTION

1. Field of the Invention

The present invention relates to mutant proteinase inhibitors and fragments thereof, including serpins (serine proteinase inhibitors), particularly inhibitory serpins such as plasminogen activator inhibitors (PAIs), anti-thrombin III, and α₁-antitrypsin (α₁AT). The proteinase inhibitors in question comprise an amino acid sequence with at least one epitope in which a mutation has altered the binding of the mutant molecule to an anti-proteinase inhibitor antibody, relative to the binding of a corresponding wild-type molecule to the same antibody. The invention also relates to the use of a mutant proteinase inhibitor or fragment thereof for screening or designing proteinase inhibitor-inactivating agents or compounds that have potential as therapeutic agents to treat conditions associated with increased levels of proteinase inhibitors in vivo. The invention more broadly relates to the use of such technology to identify tertiary binding epitopes in metastable proteins that are not amenable to rational drug design screening, thereby to screen for and design inhibitor compounds or agents that have potential to reduce levels of such metastable proteins in the body.

2. Description of the Related Art

Considerable information is available on proteinase inhibitors and their ability to regulate many biologically important systems in the body. The influence of these inhibitors is based on their ability to regulate a variety of divergent proteinases. Particularly important proteinase inhibitors are serpins, a super family of inhibitors that apparently share a common tertiary structure, Doolittle (1983), Science 222: 417-419, and may have a common evolutionary ancestor. Hunt et al. (1980), Biochem. Biophys. Res. Comm. 95:864-871. Proteins with recognizable sequence homology have been identified in vertebrates, plants, insects and viruses but not, thus far, in prokaryotes. Huber et al., (1989). Biochem. 28: 8951-8966; Sasaki (1991), Eur. J. Biochem. 202:255-261; Komiyama et al. (1994) J. Biol. Chem. 269: 19331-19337. Current models of serpin structure are based largely on seminal X-ray crystallographic studies of one member of the family, α₁-antitrypsin (α₁AT). Huber et al., (1989),. supra. Loebermann and co-workers solved the structure of a modified form of α₁AT, cleaved in its reactive center, by proposing a model where the native reactive center is part of an exposed loop, also called the strained loop. See Loebermann et al., (1984). J. Mol. Biol. 177: 531-557; Carrell et al. In PROTEINASE INHIBITORS 403-20 (Elsevier Science Publishers, 1986); Sprang, S. R. (1992). Trends Biochem. Sci. 17: 49-50.

Recent crystallographic structures of several native serpins, with intact reactive center loops, are consistent with Loebermann's hypothesis that the overall native serpin structure is very similar to cleaved α₁AT, but that the reactive center loop is exposed above the plane of the molecule. Schreuder et al., (1994) Nature Structural Biology 1: 48-54; Carrell et al. (1994) Structure 2: 257-270; Stein et al. (1990) Nature 347: 99-102; Wei et al., (1994) Nature Structural Biology 1: 251-258.; Sharp et al., (1999) Structure 7:111-118. Additional evidence has come from studies where synthetic peptides, homologous to the reactive center loops of α₁AT, antithrombin III (ATIII), or plasminogen activator inhibitor-1 (PAI-1), when added in trans, incorporate into their respective molecules, presumably as a central strand of β-sheet A. Björk, et al. (1992), J. Biol. Chem. 267, 19047-19050; Björk, I. (1992), J. Biol. Chem. 267, 1976-1982; Schulze et al. (1990), Eur. J. Biochem. 194: 51-56; Carrell et al. (1991), Nature 317:730-732; Kvassman et al. (1995) Bichem. 37: 15491-15502. This leads to an increase in thermal stability similar to that observed following cleavage of a serpin at its reactive center, and converts the serpin from an inhibitor to a substrate for its target proteinase.

An additional serpin structural form has also been identified, the so-called latent conformation. In this structure the reactive center loop is intact, but instead of being exposed, the entire amino-terminal side of the reactive center loop is inserted as the central strand into β-sheet A. Mottonen et al. (1992) Nature 355:270-273. This accounts for the increased stability of latent PAI-1 (Lawrence et al.(1994b) Biochem. 33: 3643-3648), as well as its lack of inhibitory activity (Hekman et al. (1985) J. Bio. Chem. 260:11581-11587). The ability to adopt this conformation is not unique to PAI-1, and has also now been shown for ATIII and α₁AT. Carrell et al. (1994), supra; Lomas et al. (1995) J. Bio. Chem. 270:5282-5288. Together, these data have led to the hypothesis that active serpins have mobile reactive center loops, and that this mobility is essential for inhibitor function. Carrell et al. (1991), supra; Carrell et al. (1992), Curr. Opin. Struct. Biol. 2: 438-446; Lawrence et al. (1994a) J. Bio. Chem. 269:27657-27662; Shore et al. (1994) J. Bio. Chem. 270:5395-5398; Lawrence et al. (1995) J. Bio. Chem. 270:25309-25312; Fa et al.(1995) Biochem. 34: 13833-13840; Olson et al. (1995) J. Bio. Chem. 270:30007-30017; Lawrence et al. (1990) J. Bio. Chem. 265:20203-20301.

An important member of the serpin super-family is plasminogen activator inhibitor (PAI). The PAIs have become recognized as critical regulators of the plasminogen activator (PA) system. The identification of an efficient inhibitor of t-PA in endothelial cells was first reported by Loskutoff et al., (1983) Proc. Natl. Acad. Sci. USA 80:2956-2960. Four kinetically relevant PAIs are recognized currently: PAI-1, initially described as the “endothelial cell PAI,” PAI-2, also referred to as “placental PAI,” PAI-3, also known as “activated protein C-inhibitor,” and proteinase nexin 1 (PN-1), also called “glia-derived neurite-promoting factor.”

Recent interest has centered on PAI-1 because it plays an important role in fibrinolysis and is an established risk factor for cardiovascular disease. PAI-1 is the major plasminogen activator (PA) inhibitor in plasma and platelets. Booth et al., (1988) Br. J. Haematol. 70:327-333; Fay et al.(1992) N. Engl. J. Med. 327:1729-1733; Fay et al.(1994) Blood 83:351-356.

The PAI-1 gene is 12.3 kb in length, and yields two mRNA species of 2 kb and 3 kb that both encode the same 50 kDa single-chain glycoprotein. Ny et al. (1986) ) Proc. Natl. Acad. Sci. USA 83:6776-6780; Strandberg et al. (1988), Eur. J Biochem. 176: 609-616; van Mourik et al. (1984) J. Bio. Chem. 259:14914-14921. PAI-1 is the most efficient inhibitor known of both uPA and tPA. Lawrence et al. (1989), Eur. J. Biochem. 186: 523-533; Sherman et al. (1992) J. Bio. Chem. 267:7588-7595.

PAI-1 exists in three interconvertible conformations: an active, a latent and a substrate form. The active conformation inhibits its target proteinases, tissue- type plasminogen activator (tPA) and urokinase-type plasminogen activator (uPA), by the formation of stable covalent complexes. The reactive site bond (P-P′₁) is inaccessible to the target proteinases in the latent form whereas the non-inhibitory substrate form of PAI-1 is cleaved at the reactive site bond by the serine proteinases, resulting in an irreversible inactivation of PAI-1, and with the regeneration of the proteinase activity.

Active PAI-1 decays to the latent form with a half-life of approximately 1 hour at 37° C. With exposure to denaturants (guanidine HCl or SDS), latent PAI-1 can be returned partially to the active form. Though recent X-ray crystallographic findings suggest a structural basis for these two conformations (Mottonen et al. (1992), supra), their biological significance remains unknown. Negatively-charged phospholipids can convert latent PAI-1 to the active form, suggesting that cell surfaces may modulate PAI-1 activity Lambers et al. (1987) J. Bio. Chem. 262:17492-17496. The observation that latent PAI-1 infused into rabbits is apparently converted to the active form is consistent with this hypothesis Vaughan et al. (1990) Cir. Res. 67:1281-1286. Kinetic and other evidence has also been presented for a second site of interaction between PAI-1 and tPA, outside of the PAI-1 reactive center. Lawrence et al. (1990), supra; Hekman, et al. (1988). Arch. Biochem. Biophys. 262,199-210.

PAI-1 plays an important role in the fibrinolytic system, in which it reduces the endogenous ability to remove fibrin by inhibiting plasminogen activators (PAs), such as tissue type PA (tPA) and urokinase-type plasminogen activator (uPA). Studies also have shown that elevations of PAI-1 are associated with increased risk for thromboembolic disease. Hamsten et al.(1985) N. Engl. J. Med. 313:1557-1563; Krishnamurti et al. (1992) Semin. Thromb. Hemost. 18:67-80; Schneiderman et al. (1992) Proc. Natl. Acad. Sci. USA 89:6998-7002. Therefore, inactivation of PAI-1 would potentially be of great therapeutic value. Levi et al. (1994) Blood 83: 351-356; Fay et al. (1995) Circulation 91: 1175-1181.

Strategies for reducing PAI activity in vivo fall into two basic categories. First, PAI-1 synthesis can be reduced directly through the action of drugs that depress PAI-1 gene expression. Second, PAI-1 activity can be blocked by specific antibodies or by pharmacological agents that act as specific inhibitors of PAI-1. Direct inactivators of PAT-1 have the potential for specifically reducing the total PAI-1 activity in plasma, and several recent reports demonstrated their efficacy. Levi et al., supra; Biemond et al. (1995) Circulation 91: 1175-1181; Stringer et al. (1994) Arterioscler. Thromb. 14: 1452-1458; Charlton et al. (1997) Fibrinolysis & Proteolysis 11: 51-56; Friderich et al. (1997) Circulation 96: 916-921; van Giezen et al. (1997) Thromb. Haemost 77: 964-969.

In the first studies to use this approach, PAI-1 activity was reduced by reaction with specific anti-PAI-1 monoclonal antibodies. Levi et al., supra; Biemond et al., supra; Stringer et al., supra; Debrock et al. (1997) Biochim. Biophys. Acta (1997) 1337:257-266; Debrock et al. (1997) Gene (1997) 189: 83-88. These studies demonstrate that anti-PAI-1 antibodies can be effective at neutralizing PAI-1 activity both in vitro and in vivo, and suggest that in circumstances when acutely high PAI-1 levels may be detrimental, such therapies may be useful, as for example, as an adjunct to thrombolytic therapy. Although antibody therapy looks promising, its long-term use is unlikely to be successful.

A related strategy for therapy that shows more promise is to prevent or at least influence reactive center loop insertion during PAI-1 interaction with PAs, and thereby prevent formation of the stable covalent complex. This method of blocking PAI-1 activity has been reported to occur in at least two different systems. In the first system, a synthetic peptide analogous to the reactive center loop of PAI-1 inserts into the β-sheet, and once there, it prevents the efficient insertion of the natural loop upon cleavage by a PA. Kvassman et al. (1995) J. Biol. Chem. 270: 27942-27947. As a consequence, PAI-1 is converted to a substrate for PAs, effecting an irreversible inactivation of PAI-1 by a PA. In a second study, a longer peptide of related sequence was also shown to inactivate PAI-1 Eitzman et al. (1995) J. Clin. Invest. 95:2416-2420. But this peptide seemed to induce a conformational change in PAI-1 that did not convert it to a substrate but instead to the non-inhibitory latent form.

Conventional technology has not afforded large scale and relatively simple methodology or selecting potentially effective compounds to treat cardiovascular disease and other pathological conditions that are characterized by elevated levels of a proteinase inhibitor, particularly PAI-1.

### SUMMARY OF THE INVENTION

The present invention addresses the need to identify specific proteinase inhibitor inactivating agents by disclosing an assay that can be used to screen for them. This assay utilizes the recognition that specific antibodies bind to proteinase inhibitors, specifically serpins, more specifically PAIs, α₁AT (Whisstock et al., (2000) J. Mol. Biol. 296(2): 685-699), and anti-thrombin III, (Larsson et al., (2000) Cancer Res. 60(23): 6723-6729), and most specifically PAI-1. This recognition led the present inventors to study proteinase inhibitors by mapping the epitopes to which specific anti-proteinase inhibitor antibodies bind. Locating these epitopes in the proteinase inhibitors and mutating these eptiopes results in the production of specific mutant proteinase inhibitors and fragments thereof that are used for the design and screening of compounds that inhibit specific proteinase inhibitors. The present disclosure provides an assay suitable for screening inhibitors of any metastable protein which cannot be studied using conventional methodology, such as a traditional epitope-identification screen that utilizes synthetic peptides, or a lambda gt11 phage screen or a protein crystal formation.

The present invention is directed to a mutant proteinase inhibitor comprising a wild-type proteinase inhibitor amino acid sequence with at least one mutation in at least one epitope of said amino acid sequence, wherein the mutation alters the binding of the mutant proteinase inhibitor to an anti-proteinase inhibitor antibody as compared to the binding of the wild-type proteinase inhibitor to the anti-proteinase inhibitor antibody.

The present invention is additionally directed to a fragment of the mutant proteinase inhibitor, wherein the fragment comprises at least one mutation in at least one epitope of the amino acid sequence of a wild-type proteinase inhibitor fragment.

The present invention further is directed to a nucleic acid sequence comprising a nucleotide construct encoding the mutant proteinase inhibitor or a fragment of the mutant proteinase inhibitor comprising at least one mutation in at least one epitope of the amino acid sequence.

The present invention additionally is directed to a vector and host transformed or transfected with the nucleic acid construct or vector comprising the nucleic acid sequence or construct encoding the mutant proteinase inhibitor or a fragment of the mutant proteinase inhibitor comprising at least one mutation in at least one epitope of the amino acid sequence.

The present invention further is directed to a method for producing a mutant proteinase inhibitor or a fragment thereof comprising culturing the host transformed or transfected with the nucleic acid construct or vector comprising the nucleic acid sequence or construct encoding the mutant proteinase inhibitor or a fragment thereof, under conditions wherein said nucleic acid sequence is expressed.

The present invention is further directed to a method of screening at least one compound that affects the activity of a proteinase inhibitor. The method is directed to screening for at least one compound by comparing the extent of binding of the compound to the mutant proteinase inhibitor or the fragment thereof to the binding to the wild-type proteinase inhibitor or fragment thereof.

The present invention is additionally directed to a method of screening at least one compound that affects the activity of a proteinase inhibitor, particularly the inhibitory activity of the proteinase inhibitor. The method is directed to screening for at least one compound that has less of an effect or no effect on the inhibitory activity on the inhibitory activity of the mutant proteinase inhibitor or the fragment thereof as compared to the effect on the inhibitory activity of the wild-type proteinase inhibitor or fragment thereof.

In a further embodiment, the present invention is additionally directed to a method of mapping compound binding sites in a metastable protein by comparing the effect of the compound on a measurable activity of the mutant metastable protein of fragment thereof to the measurable activity of the wild-type metastable protein or fragment thereof.

### BRIEF DESCRIPTION OF THE DRAWINGS

FIG. 1a-1d show the nucleotide sequence encoding human PAI-1 with 5′ and 3′ untranslated regions from a specific clone. Also shown is the amino acid sequence of the full length human PAI-1 including the signal sequence.

FIG. 2 provides a list of specific site-directed mutations generated in the human PAI-1 molecule in order to map the MA-33B3 monoclonal antibody binding site on human PAI-1.

FIG. 3 provides a table summarizing 11 independent human PAI-1 clones showing the observed amino acid substitutions in the amino acid sequence shown in FIG. 1 and corresponding functional activity of these mutant clones.

FIG. 4 is MA-33B8-induced inactivation of wild-type and mutant human PAI-1. ●=wild-type PAI-1; ▴=141B; ♦=S331R and ▪=D89G

FIG. 5 shows the real-time binding of multiple (A) and site-directed (B) hPAI-1 mutants to inactivating murine monoclonal 33B8 antibodies measured with BIAcore 3000 biosensor. Murine monoclonal 33B8 antibodies were immobilized on research grade chip CM5 (BIAcore) by amine coupling method. Samples of E. coli cell-free extracts in HBS-P buffer pH 7.4 (BIAcore) containing 62 nM active PAI-1 were injected in HBS-P buffer at flow rate 20 μl/minute. The association and dissociation were observed in real time over period of 5 minutes. Panel A: 1-wt hPAI-1; 2-mm1; 3-mm1M; 4-mm3; 5-mm3M; 6-mm4M; 7-mm5; 8-mm7; 9-mm8; 10-mm9; 11-mm10. Panel B: 1-wt hPAI-1; 2-N87Dsdm; 3-K88Esdm; 4-D89Gsdm; 5-Q174Rsdm; 6-G230Vsdm; 7-T232Ssdm; 8-N329Isdm; 9-S331Rsdm.

FIG. 6 shows eight site-directed mutations that were constructed to create PAI-1 variants that are specifically defective in binding of the monoclonal antibody 33B8, along with the specific nucleotide replacements made and the mutagenic primers used to introduce these replacements.

### DETAILED DESCRIPTION OF THE PREFERRED EMBODIMENT

The present invention embodies an approach to screening for compounds that bind to or affect the activity of metastable proteins. In this context, a “metastable” protein is an unstable protein that is not amenable to co-crystallization with a compound, followed by a determination of the coordinates of this binding event. Thus, a metastable protein is not readily subject to rational drug design. A metastable protein within the meaning of the present invention and to persons skilled in the art is a protein that is folded into a conformation that is not its most thermodynamicly stable conformation.

Representing an advance over known epitope-identification techniques, the approach of the present invention maps the binding site(s) by screening for loss of antibody binding to specific members within libraries of mutants of metastable proteins. This binding may be determined by some measurable activity possessed by the wild-type metastable protein or its mutant. Mutants that lose only their ability to bind to the antibody, and not other functions of the wild-type metastable protein, identify the binding site(s) as encompassing the various amino acid residues that are present only in these specific mutants.

Accordingly, the present invention encompasses screening compounds for the ability to inhibit a protein that is metastable and, hence, cannot be put through a typical rational drug-design regimen, involving co-crystallization with the compound. One class of such proteins includes proteinase inhibitors, such as the serpins, including α₁ATs, antithrombins, and PAIs, particularly PAI-1. In accordance with the present invention, therefore, one can design and prepare mutants of proteinase inhibitors (that is, full-length proteins and biologically active fragments thereof) in light of information gleaned from binding studies between wild-type proteinase inhibitors and antibodies specific for proteinase inhibitors.

The drug screening approach of the present invention exploits the changes (mutations) in the mutant proteinase inhibitors to identify compounds that bind less strongly to, that inactivate to a lesser degree, or that cannot inactivate the mutant proteinase inhibitors, but that bind more strongly to, that can inactivate to a greater degree, or that can inactivate wild-type proteinase inhibitor. For example, there are multiple potential binding sites for compounds on PAI-1. If a single, specific compound effects wild type PAI-1 inhibition activity but cannot inhibit the mutant PAI-1, then it is reasonable to conclude that the compound is binding at the mutated site in the PAI-1. From this information, additional compounds can be designed based upon the structure of this “inactive” molecule. Thus, rationale drug design accordingly to the present invention can proceed without having the protein crystallized. Moreover, while the present description discusses PAI-1 as a representative metastable protein, the aforementioned drug-screening methodology has impact outside of the PAI-1 field.

The activity measured as a result of the interaction of the mutant protein must be compared to the activity of the corresponding wild-type protein's interaction with the compound. For example with PAI-1, it is inherent in the assay that both the mutant and wild type PAI-1 have equivalent inhibitory activity against the target proteinase, prior to mixing the PAI-1 with the compound. Alternatively, control runs can be performed to take into account any differences in inhibitory activity of the mutant and wild-type PAI-1s.

A mutant proteinase inhibitor obtained according to the present invention can be used in screening for and designing compounds to affect proteinase inhibitors in vivo, thereby to realize therapeutic potential in treating diseases that involve elevated proteinase-inhibitor levels. Such mutant proteinase inhibitors also can be employed to identify specific antibodies or other binding epitopes that are tertiary in nature and not revealed by traditional epitope identification techniques, Which utilize synthetic peptides or a lambda gt11 phage screen or which require protein crystal formation.

To develop a reliable screen for metastable proteins, the inventors have drawn from their expertise with a serpin, PAI-1, and the binding interaction between PAI-1 and anti-PAI antibodies. Specifically, the inventors measured the binding between mutant and wild-type PAI-1 and anti-PAI antibodies, and compared the extent of binding of the anti-PAI antibodies to mutant and wild-type PAI-1s. The inventors observed that certain PAI-1 mutants lost their ability to bind (or they bound at a reduced level) to an anti-PAI-1 antibody, relative to binding to and inactivation of wild-type PAI-1. For the disclosed method to function as a viable screen, all that is necessary is that this relative binding between the mutant and wild-type PAI-1s be measurable. Further, the method of screening can also be based on measuring some activity of the metastable protein, PAI-1. Preferably, PAI-1 inhibitory activity against its target proteinase is the activity that is measured, following contact with anti-PAI antibodies but any other measurable activity of PAI-1 can be used in the screening method.

Another embodiment of the present invention relates to PAI-1 mutants that contain one or more epitope mutations, relative to a corresponding wild-type PAI-1, that are involved in binding an anti-PAI antibody. The key to the screening method of the present invention is that the mutation(s) in the PAI-1 mutant are used to identify a compound that binds less strongly to the mutant PAI-1 than the same compound binds to the corresponding wild-type PAI-1. This wild-type PAI-1 contains the same amino acid sequences as the mutant PAI-1 except that the epitope to which the anti-PAI antibody binds in the wild-type PAI-1 is modified to contain one or more changes, such as a substitution, a deletion and/or an addition of at least one amino acid that make up the epitope. The epitope is modified to produce mutant PAI-1s of the present invention. Although the present invention encompasses any modification of the epitope(s) in the mutant PAI-1, amino acid substitutions in the epitope are preferred. The mutants are prepared according to the present invention and are tested for activity and binding to anti-PAI antibodies, thereby to determine their utility in the screening method. In this regard, the term “epitope” connotes a specific site of interaction or binding between two molecules, and it generally takes on an immunological definition to describe, for example, the binding between an anti-PAI antibody and a mutant or wild-type PAI-1, where the definition of an epitope is the antigenic determinant on an antigen to which the paratope, the specific site within an antibody Fab region, binds.

The screening method of the present invention can measure binding to the mutant and wild-type PAI-1s of compounds that are to be screened. The extent of binding between compounds and the mutant and wild-type PAI-1s are compared and compounds that bind less to the mutant PAI-1 than to the wild-type PAI-1 are selected as potential compounds that affect the inhibitory activity of PAI-1.

More particularly, the mutant PAI-1s are used in a screening method for evaluating compounds for their effect on mutant and wild-type PAI-1 activity. A compound that has potential as a PAI-1 inhibitory compound affects mutant PAI-1 activity less than it affects the wild-type PAI-1 activity. The PAI-1 activity that is measured is preferably PAI-1's inhibitory activity against its target proteinase, and therefore the screening method preferably measures the effect on the PAI-1 inhibitory activity of the mutant PAI-1 as compared to the effect on the PAI-1 inhibitory activity of the wild-type PAI-1. More particularly, compounds that do not affect or inactivate the mutant PAI-1 activity but do affect to a greater extent or inactivate the corresponding wild-type PAI-1 are compounds that the screening method is designed to identify. The most preferred compounds that are identified by this method are those compounds that do not bind to the mutant proteinase inhibitor or a fragment thereof but do bind to the wild-type proteinase inhibitor or a fragment thereof.

There are multiple potential binding sites for compounds on PAI-1. If a specific compound inhibits the wild-type PAI-1 but cannot inhibit the mutant PAI-1, then the compound binding site must be the mutated epitope to which the specific compound binds.

From this information, additional compounds are designed to bind to the wild-type PAI-1 based upon the structure of the compound that is inactive against the mutant PAI-1. For example, if the identified epitope of the anti-PAI-1 antibody overlaps with a binding pocket for one or more compounds, such as compounds that inactivate PAI-1, then the PAI-1 mutants disclosed in the present invention provide a useful screen for PAI-1 inactivating agents.

Additionally, the screening method of the present invention is useful for identifying specific antibodies or other binding epitopes that are tertiary in nature, and therefore, refractive to traditional epitope identification screens, such as those using synthetic peptides or a lambda gt11 phage screen. Further, the screening method is useful to identify other proteins that may bind differentially to mutant PAI-1s.

The present invention also is directed to a method of screening at least one compound that affects the activity of a metastable protein, which by definition is unstable and not amenable to rational drug design. The method also is useful for mapping compound binding sites in a metastable protein.

Thus, in one embodiment the present method comprises (a) incubating a compound separately with: (i) a mutant metastable protein or a fragment thereof, and (ii) a wild-type metastable protein or a fragment thereof; (b) measuring the effect of the compound in (a) on a measurable activity of the mutant metastable protein or a fragment thereof and the wild-type metastable protein or fragment thereof; and (c) comparing the effect of the compound in (b) on the measurable activity of the mutant metastable protein or the fragment with the effect of the compound on the measurable activity of the wild-type metastable protein or the fragment thereof. When the compound affects the measurable activity of the wild-type stable protein, the method further comprises: (d) selecting a compound that has less of an effect on the activity of the mutant metastable protein or the fragment thereof in (b) than the effect on the activity of the wild-type stable protein or the fragment.

More specifically, the present invention is directed to a method of mapping at least one compound binding site in a metastable protein or fragment thereof comprising: (a) incubating a mutant metastable protein or fragment thereof with a compound; (b) measuring the effect of the compound in (a) on a measurable activity of the mutant metastable protein; (c) performing a parallel set of steps (a′) and (b′) comprising the same steps as (a) and (b), except that the wild-type metastable protein is substituted for the mutant metastable protein in step (a′); (d) comparing the effect of the mutant metastable protein in (b) and the wild-type metastable protein in (b′); and (e) selecting a compound that has less of an effect on the measruable activity of the mutant metastable protein or fragment thereof in (b) than the effect on the measurable activity of the wild-type metastable protein or fragment in (b′).

The inventive screening method also may entail control treatments comprising: (f) performing a parallel set of steps (a″) through (d″) corresponding to steps (a) through (d) and performing a parallel set of steps (a′″) through (d′″) corresponding to steps (a′) through (d′), except that no compound is added in step (a) or (a′); and (g) comparing the measurable activity of the mutant metastable protein in (b) and (b″) and the wild-type metastable protein in (b′) and (b′″) to determine the relative effect on a measurable activity of the mutant metastable protein and the wild-type metastable protein.

The method further comprises performing a control incubation, wherein the measurable activity is measured without the addition of the mutant metastable protein or the wild-type metastable protein in (a) and (a′), described above. The method further comprises performing a control incubation, wherein the measurable activity is measured without the addition of the mutant metastable protein, the wild-type metastable protein or the compound in (a) and (a′). The method additionally comprises a mutant metastable protein or a fragment thereof that is prepared by introducing at least one mutation in at least one epitope of the amino acid sequence of said wild-type metastable protein or a fragment thereof, wherein the mutation alters the binding of the mutant metastable protein or fragment thereof to an antibody that binds to the wild-type metastable protein or fragment thereof.

A mutant metastable protein or fragment thereof, used in the method described above, is prepared by introducing at least one mutation in at least one epitope of the amino acid sequence of the wild-type metastable protein or fragment thereof, wherein the mutation alters the binding of the mutant metastable protein or fragment thereof to an antibody that binds to a greater extent to the wild-type metastable protein or fragment thereof than to the mutant metastable protein.

The mutant metastable protein or fragment thereof of the present invention retains from about 5-100% of its measurable activity in the presence of the compound, and more preferably retains at least about 25% of its measurable activity in the presence of the compound. More preferably the mutant metastable protein or fragment thereof of the present invention retains at least about 50% of its measurable activity in the presence of the compound, and more preferably retains at least about 75% of its measurable activity in the presence of the compound. Most preferably the compound has substantially no effect on the inhibitory activity of the mutant metastable protein or fragment thereof, and the metastable protein. The retention of measurable activity as indicated above were measured in the presence of the compound.

The present invention is more preferably directed to a method of screening at least one compound that affects the activity of a proteinase inhibitor comprising: (a) incubating a compound with: (i) a mutant proteinase inhibitor as described herein or a fragment thereof, wherein the inhibitor or the fragment comprises at least one mutation in at least one epitope of the amino acid sequence; and separately incubating the compound with (ii) a wild-type proteinase inhibitor or a fragment thereof; (b) measuring the binding of the mutant proteinase inhibitor or the fragment and the wild-type proteinase inhibitor or the fragment with the compound; and (c) comparing the binding of the compound to the mutant proteinase inhibitor or the fragment with the binding of the compound to a wild-type proteinase inhibitor or the fragment. The method further comprises: (d) selecting a compound that binds less strongly with the mutant proteinase inhibitor or the fragment thereof as compared to the binding of the compound to the wild-type proteinase inhibitor or the fragment.

A compound that binds to the mutant proteinase inhibitor or the fragment thereof about 95% or less than the binding of the compound to the wild-type proteinase inhibitor or fragment thereof is preferred, and a compound that binds to the mutant proteinase inhibitor or the fragment thereof about 75% or less than the binding of the compound to the wild-type proteinase inhibitor or fragment thereof is more preferred. A compound that binds to the mutant proteinase inhibitor or the fragment thereof about 50% or less than the binding of the compound to the wild-type wild-type proteinase inhibitor or fragment thereof is more preferred, and a compound that binds to the mutant proteinase inhibitor or the fragment thereof about 24% or less than the binding of the compound to the wild-type proteinase inhibitor or fragment thereof is more preferred. But a compound that does not bind to the mutant proteinase inhibitor or the fragment thereof but does bind to the wild-type proteinase inhibitor or the fragment thereof is most preferred.

In addition to screening compounds by measuring binding of the compound to the mutant and wild-type proteinase inhibitors, the method of screening further can measure the effect of at least one compound on a measurable activity of a proteinase inhibitor. In this aspect, the method comprises: (a) incubating a compound with: (i) a mutant proteinase inhibitor comprising a wild-type mutant proteinase inhibitor amino acid sequence with at least one mutation in at least one epitope of the amino acid sequence, wherein the mutation alters the binding of the mutant proteinase inhibitor to an anti-proteinase inhibitor antibody as compared to the binding of the wild-type proteinase inhibitor to the anti-proteinase inhibitor antibody or a biologically active fragment of the mutant proteinase inhibitor, wherein the inhibitor or the fragment comprises at least one mutation in at least one epitope of the amino acid sequence; and separately incubating the compound with: (ii) a wild-type proteinase inhibitor or a fragment thereof; (b) measuring the effect of the compound in (a) on the inhibitory activity of the mutant proteinase inhibitor or the fragment thereof and the wild-type proteinase inhibitor or the fragment thereof; and (c) comparing the effect of the compound on the mutant proteinase inhibitor or the fragment thereof with the effect of the compound on a wild-type proteinase inhibitor or the fragment. When the compound affects the inhibitory activity of the wild-type proteinase, the method further comprises: (d) selecting a compound that has less of an effect on the inhibitory activity of the mutant proteinase inhibitor or the fragment in (b) than the effect on the inhibitory activity of the wild-type proteinase inhibitor or the fragment.

Mutant proteinase inhibitors and fragments thereof that retain from about 5-100% of its inhibitory activity in the absence of the compound is also useful in the present invention, and mutant proteinase inhibitors and fragments thereof that retain at least about 25% of its inhibitory activity is more preferred. But mutant proteinase inhibitors and fragments thereof that retain at least about 50% of its inhibitory activity is more preferred, and mutant proteinase inhibitors and fragments thereof that retain at least about 24% of its inhibitory activity is more preferred. The most preferred compounds that are identified by this method are those compounds that have no inhibitory effect on the mutant proteinase inhibitor or a fragment thereof but do inhibit the wild-type proteinase inhibitor or a fragment thereof, where the mutant proteinase inhibitor or fragment thereof retains 100% of its inhibitory activity. The retention of the inhibitory activity of the mutant proteinase inhibitor or fragment thereof against its target proteinase as indicated above is measured in the absence of the compound.

More preferably, the method of the present invention comprises screening compounds that affects a mutant proteinase inhibitor comprising: (a) incubating a mutant proteinase inhibitor comprising a wild-type proteinase inhibitor amino acid sequence with at least one mutation in at least one epitope of the amino acid sequence, wherein the mutation alters the binding of the mutant proteinase inhibitor to an anti-proteinase inhibitor antibody as compared to the binding of the wild-type proteinase inhibitor to the anti-proteinase inhibitor antibody or a fragment thereof with a compound; (b) measuring the effect of the compound in (a) on the inhibitory activity of the mutant proteinase inhibitor on a target proteinase; (c) performing a parallel set of steps (a′) and (b′) comprising the same steps as (a) and (b), except that wild-type proteinase inhibitor is substituted for the mutant proteinase inhibitor in step (a′); (d) comparing the effect on the inhibitory activity of the mutant proteinase inhibitor in (b) and the wild-type proteinase inhibitor in (b′), and (e) selecting a compound that has less of an effect on the inhibitory activity of the mutant proteinase inhibitor in (b) than on the wild-type proteinase inhibitor in (b′).

In regard to the measuring of step (b) and (b′), the method more preferably comprises adding a target proteinase to the mutant proteinase inhibitor and the compound of (a) to form a first mixture and to the wild-type proteinase inhibitor and the compound of (a′) to form a second mixture and incubating the first and second mixtures. The measuring of (b) and (b′) comprises measuring the target proteinase activity that comprises adding a substrate of the target proteinase and measuring the enzymatic conversion of the substrate.

The method of screening further comprises control treatments comprising: performing a parallel set of steps (a″) through (d″) corresponding to steps (a) through (d) and performing a parallel set of steps (a′″) through (d′″) corresponding to steps (a′) through (d′), except that no compound is added in step (a″) or (a′″); and (g) comparing the inhibitory activity of the mutant proteinase inhibitor in (b) and (b″) and the wild-type proteinase inhibitor in (b′) and (b′″) to determine the relative inhibitory activity of the mutant proteinase inhibitor and the wild-type proteinase inhibitor. The method further comprises the measuring step of (b″) and (b′″) comprises adding a target proteinase to the mutant proteinase inhibitor of (a″) to form a third mixture and to the wild-type proteinase inhibitor of (a′″) to form a fourth mixture, and incubating the third and fourth mixtures. The method, wherein the measuring step of (b″) and (b′″) comprises measuring the target proteinase activity, comprises adding a substrate of the target proteinase and measuring the enzymatic conversion of the substrate.

The method further comprises performing a control incubation, wherein the target proteinase activity is measured without the addition of the mutant proteinase inhibitor or the wild-type proteinase inhibitor in (a) and (a′).

The method further comprises performing a control incubation, wherein the target proteinase activity is measured without the addition of the mutant proteinase inhibitor, the wild-type proteinase inhibitor and/or the compound in (a) and (a′).

The present invention is directed to the mutants that are used in the compound screening method(s) of the present invention. The mutant proteinase inhibitors of the present invention comprises a wild-type proteinase inhibitor amino acid sequence with at least one mutation in at least one epitope of the wild-type proteinase inhibitor amino acid sequence or a fragment thereof, wherein the mutation alters the binding of the mutant proteinase inhibitor to an anti-proteinase inhibitor antibody as compared to the binding of the wild-type proteinase inhibitor to the anti-proteinase inhibitor antibody.

Preferably, the mutant proteinase inhibitor has a lower binding affinity to the antibody than the anti-proteinase inhibitor antibody has for the wild-type proteinase inhibitor. More preferably, the mutant proteinase inhibitor antibody does not bind to the mutated epitope of the mutated proteinase inhibitor.

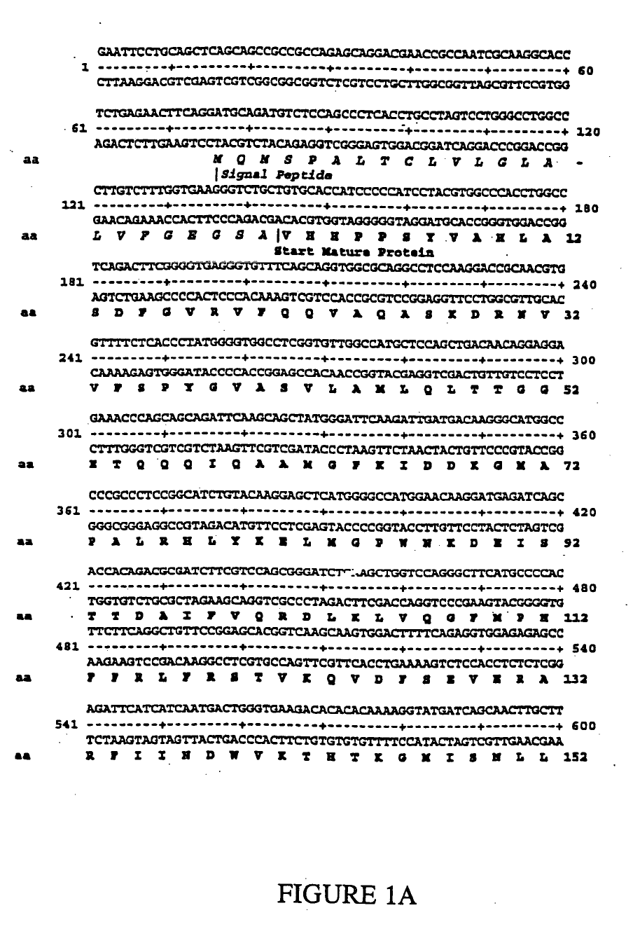

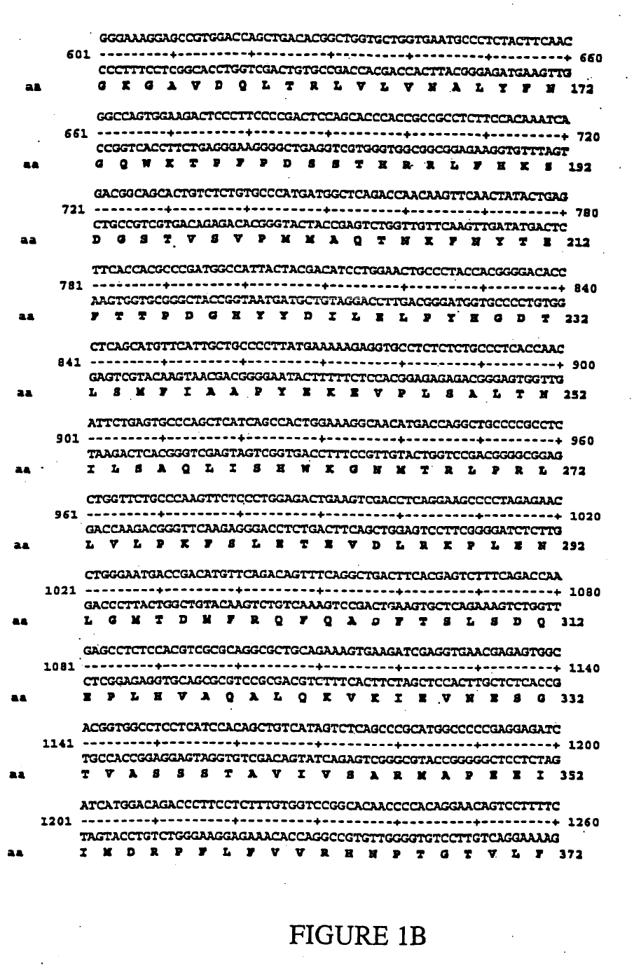

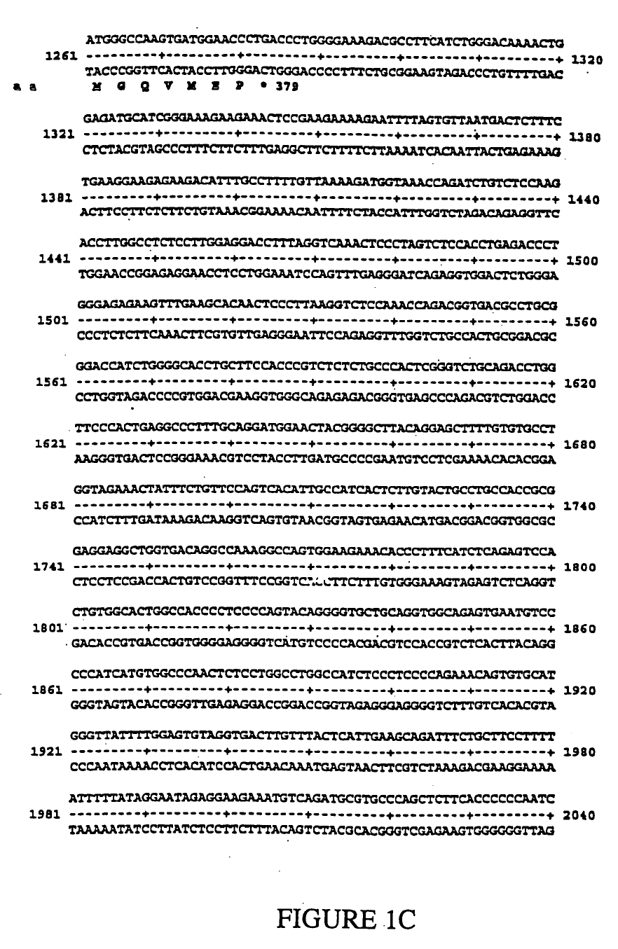

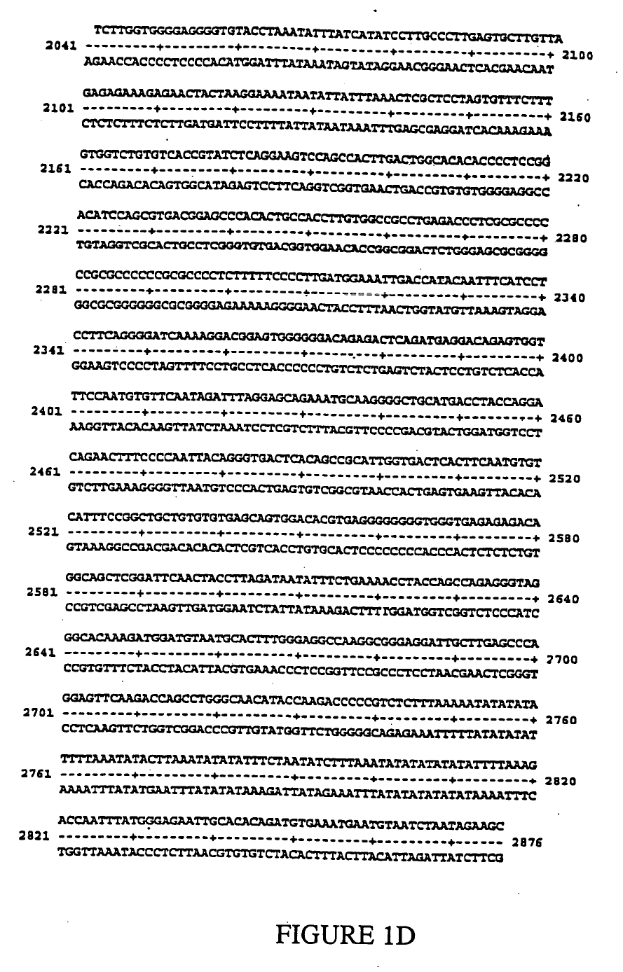

The mutant proteinase inhibitor is a mutant of the wild-type plasminogen activator inhibitor (PAI), and more preferably is a mutant of wild-type PAI-1. Most preferably, the mutant proteinase inhibitor comprises the amino acid sequence of the wild-type PAI depicted in FIG. 1.

A suitable anti-proteinase inhibitor antibody preferably is a monoclonal antibody that does not inhibit the mutant proteinase inhibitor or fragment thereof and does inhibit the wild-type proteinase inhibitor or fragment thereof. More preferably, the antibody is an anti-PAI monoclonal antibody, and more preferably an anti-PAI-1 monoclonal antibody. The most preferred anti-PAI monoclonal antibody includes but is not limited to murine monoclonal antibodies, MA-33B8 or 31C9, available commercially from Molecular Innovations, Inc. (Southfield, Mich.) However, any antibody that inhibits a measurable activity of the mutant proteinase inhibitor can be used, in accordance with the present invention, for identifying compounds that have potential to inhibit proteinase inhibitors.

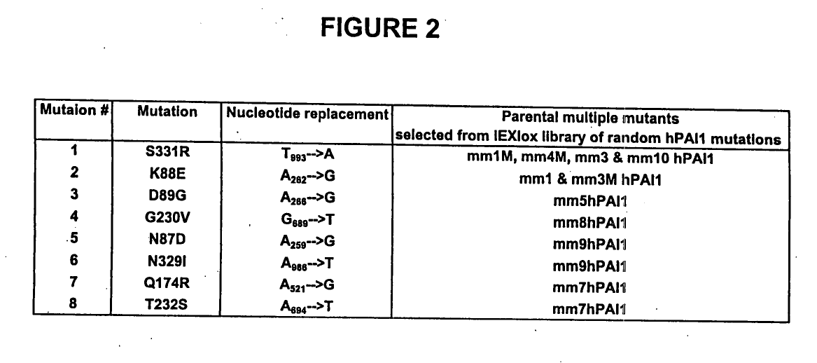

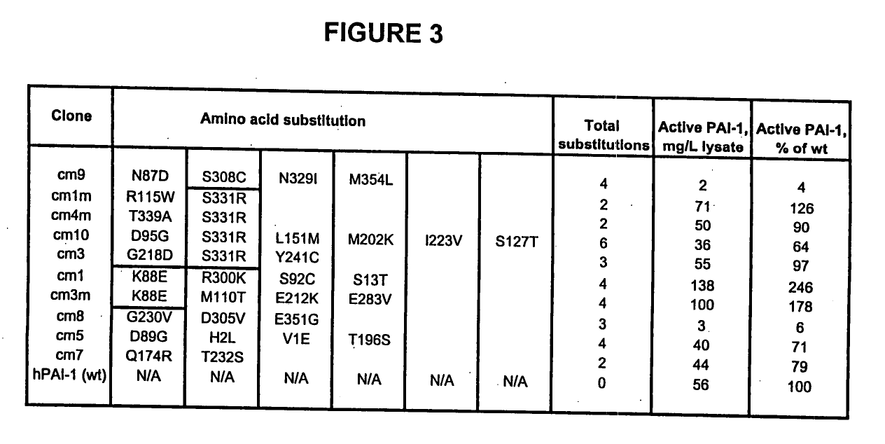

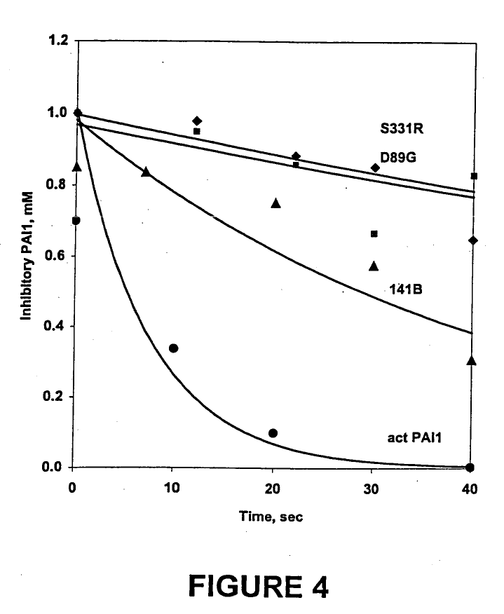

The mutant proteinase inhibitor of the present invention comprises the amino acid sequence of FIG. 1a-1d, wherein the amino acid sequence comprises a substitution at one or more amino acid residues selected from the group consisting of 1, 2, 13, 82, 87, 88, 89, 92, 95, 110, 115, 127, 151, 174, 196, 202, 212, 218, 223, 230, 241, 283, 300, 305, 308, 329, 331, 323, 339, 351 and 354. More preferably the mutant proteinase inhibitor comprises a substitution at one or more amino acid residues selected from the group consisting of 87, 88, 89, 174, 230, 232, 329 and 331. FIG. 2 discloses a table setting forth specific amino acid substitutions at these amino acid residues and FIG. 3 discloses a table setting forth PAI-1 mutants of the present invention that contain multiple substitutions.

More preferably, the mutant proteinase inhibitor of the present invention comprises at least one amino acid substitution at amino acid 87, and more preferably the amino acid substitution at amino acid 87 is changing an asparagine to an aspartic acid. The designation for such a substitution is N87D, with the wild-type amino acid recited before the amino acid number and the substituted or changed amino acid recited after the amino acid number. Additionally, another preferred mutant proteinase inhibitor comprises at least one amino acid substitution at amino acid 89, wherein the amino acid substitution at amino acid 89 is changing an aspartic acid to glycine, D89G. Another preferred mutant proteinase inhibitor comprises at least one amino acid substitution at amino acid 230, wherein the amino acid substitution at amino acid 230 is changing a glycine to a valine, G230V. Another mutant proteinase inhibitor comprises at least one amino acid substitution at amino acid 331, wherein the amino acid substitution at amino acid 331 is changing a serine to an arginine, S331R.

The present invention also encompasses a fragment of the mutant proteinase inhibitor described above. The term “fragment” is intended to be a portion of the mutant or wild-type proteinase inhibitor that is less than the full length of the proteinase inhibitor. It is important that the fragment comprises at least one mutation in at least one epitope of the amino acid sequence and it is important that when using the mutant proteinase inhibitor fragment in the screening method that its inhibitory activity be compared to the corresponding wild-type proteinase inhibitor fragment up to full-length wild-type proteinase inhibitor.

The present invention is further directed to a nucleic acid sequence comprising a nucleotide sequence encoding the mutant proteinase inhibitors described herein or a fragment of the mutant proteinase inhibitor comprising at least one mutation in at least one epitope of the amino acid sequence. The nucleic acid sequence preferably encodes a mutant of the wild-type plasminogen activator inhibitor (PAI), and more preferably is a mutant of wild-type PAI-1. Most preferably, the nucleic acid sequence encodes the mutant proteinase inhibitor comprising the amino acid sequence of the wild-type PAI depicted in FIG. 1.

The present invention also is directed to a nucleic acid construct comprising the nucleic acid sequence as described above operably linked to regulatory control sequences that effect the expression of the nucleic acid sequence. The present invention further comprises a vector comprising a nucleotide sequence encoding the mutant proteinase inhibitors described herein or a fragment of the mutant proteinase inhibitor comprising at least one mutation in at least one epitope of the amino acid sequence. The present invention is further directed to a host transformed or transfected with a nucleic acid construct of a nucleic acid sequence comprising a nucleotide sequence encoding the mutant proteinase inhibitors described herein or a fragment of the mutant proteinase inhibitor comprising at least one mutation in at least one epitope of the amino acid sequence as described above.

The mutant proteinase inhibitor or a fragment thereof of the present invention is produced by culturing a host comprising a nucleic acid sequence operably linked to regulatory control sequences that effect the expression of the nucleic acid sequence under conditions wherein said nucleic acid sequence is expressed.

To prepare mutants of wild-type proteins, a skilled person needs to know the amino acid and DNA sequences of the protein that is to be mutated.

In the case of the preferred protein, PAI-1 cDNA encodes a protein of 402 amino acids that includes a typical secretion signal sequence (Ny et al., 1986; Ginsburg et al., 1986). Mature human PAI-1 isolated from cell culture is composed of two variants of 381 and 379 amino acids in approximately equal proportions. These two forms, likely arising from alternative cleavage of the secretion signal sequence, provide proteins with overlapping amino-terminal sequences of Ser-Ala-Val-His-His and Val-His-His-Pro-Pro (see FIG. 1 and disclosed in Lawrence et al., 1989). This latter sequence is generally referred to as mature PAI-1. The complete amino acid and nucleotide sequence of PAI-1 also is disclosed in FIG. 1a-1d showing that the amino acid sequence including the 23 amino acid signal sequence is 402 amino acids in length and the mature amino acid sequenc is 379 amino acids.

PAI-1 is a glycoprotein with three potential N-linked glycosylation sites containing between 15 and 20% carbohydrate (Van Mourik et al., 1984). Mature PAI-1 contains no cysteine residues, facilitating efficient expression and isolation of recombinant PAI-1 from E. coli. PAI-1 produced in E. coli, although nonglycosylated, is functionally very similar to native PAI-1. Recombinant PAI-1 can be isolated from E. coli in an inherently active form, which contrasts with PAI-1 purified from mammalian cell culture (Lawrence et al., 1989; Hekman et al., 1988).

U.S. Pat. No. 6,103,498 discloses how to make modified PAI-1s or PAI-1 mutants and these methods are useful for preparing the mutant PAI-1s of the present invention, and is incorporated herein by reference in its entirety. Site-directed mutagenesis and other methods are used to produce and characterize a large number of mutations in the PAI-1 reactive center loop (RCL) (Sherman et al., 1992, Sherman et al., 1995). Although this previous patent application discloses how to make and identify new mutants in the RCL of PAI-1 which confer on PAI-1 new and useful properties, in particular (a) the ability to interact with and inhibit elastase, an activity which is lacking in native PAI-1 and (b) the ability to inhibit vitronectin-associated cell migration, it does not suggest preparing the mutant PAI-1s for use in the screening method of the present invention.

While the present disclosure is directed primarily to human PAI-1 or mutants thereof, it is to be understood that homologues of PAI-1 from other species, and mutants thereof, that possess the characteristics disclosed above are intended within the scope of this invention. In particular, the PAI-1 protein (or DNA) from other mammalian species may be used for the same purposes as human PAI-1 in the method of the present invention.

As noted above, the PAI-1 of the present invention can be produced recombinantly in E. coli or other host cells, or other known methods of producing proteins from DNA sequences, including the use of transgenic plants or animals using methods known to persons skilled in the art. The present invention also includes peptides that include at least that portion of the sequence which contains the substitution or substitutions, and which possess the requisite biochemical and biological activity. Such peptides are produced using well-known synthetic methods for the synthesis of polypeptides of desired sequence on solid phase supports and their subsequent separation from the support. Methods for solid phase peptide synthesis are well-described, for example, by: Merrifield, (1963); Merrifield, (1986); Wade, et al., (1986); Fields, (1990); MilliGen Report Nos. 2 and 2a (1987). For example, the “tBoc method” or an improved “F-moc” technique may be used (Atherton, et al., 1981).

### EXAMPLES

### Production of PAI-1 Mutants by Expression and Purification of Recombinant PAI-1 in E. coli

The following methods are preferred and do not represent the exclusive means for producing proteinase inhibitor mutants of the invention. Techniques for synthesizing oligonucleotide probes are well known in the art and disclosed by, for example, Wu, et al., 1978 or Gait, ed., (Current Edition). Procedures for constructing and expressing recombinant molecules in accordance with this invention, including appropriate promoters and other control elements, selection markers, etc., are disclosed by Sambrook, J. et al, 1989; Ausubel, F. M. et al., 1987, which references are herein incorporated by reference.

Included in this invention is the DNA encoding the PAI-1 mutants, which is preferably a cDNA having the appropriate nucleotide sequence substitutions to encode the mutant proteins as disclosed herein. Such molecules are prepared using conventional methods. Also included herein are prokaryotic or eukaryotic host cells transformed or transfected with a vector comprising the above DNA molecule. Again, the method used for transferring the DNA, expressing the DNA and growing the host cells are well-known in the art and described in the references cited above. Eukaryotic host cells are preferably mammalian cells of an established cell line, although insect cells or plant cells are also contemplated. Appropriate vectors such as viruses, vector sequences, control sequences, such as promoters appropriate for the species of host cells, are conventional and well-known to those skilled in the art and are therefore not described in particular detail herein. In addition to sense DNA, antisense DNA and antisense RNA molecules to the mutant PAI-1 coding sequence are provided herein. Also included is an RNA molecule encoding the PAI-1 mutant.

### Example 1

Materials

The murine anti-human PAI-1 monoclonal antibodies (mAbs) 33B8 and 31 C9, as well as rabbit anti-human PAI-1 polyclonal antibodies (pAbs) were obtained from Molecular Innovations (Southfield, Mich., USA). High molecular weight tPA and uPA were obtained from Genetech (South San Francisco, Calif.) and Molecular Innovations (Southfield, Mich., USA), respectively. Chromogenic tPA substrate pGlu-Gly-Arg p-nitroanilide was purchased from Sigma (St.Louis, Mo., USA). Nitrocellulose filters and membranes used for protein transfer were from Schleicher & Schuell, Inc. (Keene, N.H., USA).All supplies and reagents for SDS-PAGE including precast gels were from Novex (San Diego, Calif., USA). All E. coli strains used for subcloning, replication or expression of human PAI-1 wild-type or mutated genes were purchased from Stratagene (La Jolla, Calif., USA), Clontech Laboratories, Inc. (Palo Alto, Calif., USA) and Novagen (Madison, Wis., USA) as competent cells. For site-directed mutagenesis the Transformer™ Site-Directed Mutagenesis Kit was purchased from Clontech Laboratories, Inc. (Palo Alto, Calif., USA). All sequencing and mutagenic primers used were synthesized and PAGE purified by Integrated DNA Technologies, Inc. (Coralville, Iowa, USA). DNA restriction endonucleases (Sca I and Mlu I) were purchased from Life Technologies (Gaithersburg, Md., USA) and New England Biolabs, Inc.(Beverly, Mass., USA), respectively. DNA sequencing was done with the BigDye™ Terminator Cycle Sequencing Ready Reaction Kit from Applied Biosystems (Foster City, Calif., USA) on ABI PRISM™ 310 Genetic Analyzer. Analysis of DNA sequences was performed with the Vector NTI™ Suite 6.0 Molecular Biology Software for Windows™. All supplies and buffers for binding assays with BIAcore 3000™ Biosensor were purchased from Biacore AB (Uppsala, Sweden). Bovine serum albumin (BSA), sodium azide (NaN₃), isopropyl thio-β-D-galactoside (IPTG), carbanicilin, tetracyclin and kanamycin were from Sigma (St.Louis, Mo., USA). All other chemical reagents used in the study were purchased from Fisher Scientific (Pittsburg, Pa., USA).

Construction and Screening of Mutant PAI-1 Library

A library of random PAI-1 mutants with >2×10⁷ independent clones was constructed in the λ phage expression vector λEXlox by error-prone PCR as described in Berkenpas et al., 1995, incorporated by reference. Briefly, the entire coding sequence of mature PAI-1 was amplified by four cycles of error-prone PCR, as described by Lawrence et al., J. Biol. Chem. (1994) 269: 16223-16228, incorporated in its entirety by reference. The mutagenized product was gel purified from the template and re-amplified using standard PCR conditions to produce an amplified pool of randomly mutagenized PAI-1 cDNAs. The PCR product was cleaved with XbaI and EcoRI, ligated to similarly restricted λEXlox arms, and packaged with PhagePrep from Novagen (Madison, Wis., USA).

Lawns of phage-infected Escherichia coli BL21 (DE3) were prepared from exponentially grown cells in Luria broth (LB) with 0.02% maltose. To select phage plaques with mutated PAI-1 molecules, protein expression was induced by incubating pregrown phage plaques with overlaid IPTG(10 mM)+tPA(10 μg/ml) saturated nitrocellulose filters within 1 hr at 37° C. Lifted filters were once washed in TBS containing 0.5% SDS thoroughly then blocked with 1% BSA/5% milk/TBS solution and incubated with 33B8 mAbs, as a primary antibodies. Filter-bound tPA:PAI-1:33B8 mAbs complexes were detected immunologically with goat anti-mouse immunoglobulins, conjugated to horse radish peroxidase (HRP) in enhanced chemiluminescence (ECL)assay. The dark spots revealed on ECL Hyperfilms indicated plaques expressing PAI-1 molecules capable of binding to the 33B8 mAbs. Then filters were washed with water and TBS several times, incubated with rabbit anti-PAI-1 pAbs as the primary and goat anti-rabbit alkaline phosphatase (AP) conjugated antibodies as secondary ones. The dark purple spots appeared on the filters after NBT/BCIP color reaction corresponded to plaques expressing all functionally active PAI-1 molecules. To select 33B8 binding-negative plaques, the ECL Hyperfilms were overlapped with the original NBT/BCIP developed nitrocellulose filters. Plaques expressing 33B8 mAbs binding-negative phenotype were picked up from relevant bacterial plates and run through two sequential rounds of enrichment, which included the same steps as the primary screening.

Totally, three hundred thousand of λEXlox particles from L5 random mutations of h PAI-1 library were screened, and from 22 particles expressing active PAI-1 phenotype which were picked up in the first screen only 11 appeared to be positive after following two screens, and thus were isolated, automatically subcloned by Cre-mediated excision of plasmid DNA from λEXlox vector in accordance with Novagen (Madison, Wis., USA) protocol and subjected to further analysis.

DNA sequence analysis of entire PAI-1 coding region (1137 nucleotides) in 10 independent pEXmutPAI-1 plasmids demonstrated a mutational frequency of 1:366, resulting in an average of approximately three amino acid substitutions per variant.

Site-Directed Mutagenesis

Site-directed mutagenesis allows the production of peptide variants through the use of specific oligonucleotide sequences that encode the DNA sequence of the desired mutation plus a sufficient number of adjacent mucleotides to provide a primer sequence of sufficient size and sequence complexity to form a stable duplex on both sides of the deletion junction being traversed. Typically, a primer of about 20 to 30 nucleotides in length is preferred, with about 5 to 10 residues on both sides of the junction of the sequence being altered. The technique of site-directed mutagenesis is well known to persons skilled in the art, as exemplified by publications, such as Adelman et al., DNA 2:183 (1983), incorporated herein by reference.

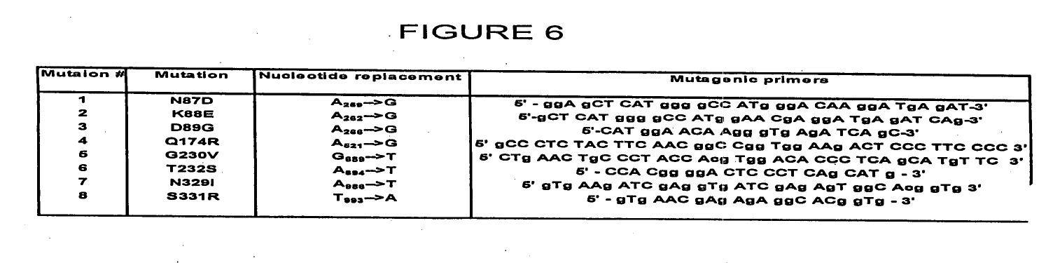

The individual PAI-1 site-directed mutants were constructed by following the protocol from Transformer™ Site-Directed Mutagenesis Kit from Clontech Laboratories, Inc. (Palo Alto, Calif., USA) but other similar kits may be used to construct the mutants. The primers used in the protocol are designed using the PAI-1 DNA sequence provided in FIG. 1 and the known mutations identified in the initial screening of the library for clones that did not bind the 33 BA monoclonal antibody as described herein. Specific mutagenic” primers that were used to introduce desired mutations into the PAI-1 DNA sequence are disclosed in FIG. 6.

After final transformation in E. coli XL-10 Gold competent cells from Stratagene (La Jolla, Calif., USA) with the mutated PAI-1 DNA sequences, several clones of each individual mutant (at least four) were selected and sequenced throughout the entire PAI-1 coding region with 3-5 sequencing primers. Eventually, eight of the site-directed mutants, with the single amino acid replacement in positions 87, 88, 89, 174, 230, 232, 329 and 331 of PAI-1 molecule were constructed and evaluated as disclosed herein.

Functional Analysis of PAI-1 Molecules

Multiple 33B8-binding negative and single amino acid replacement hPAI-1 mutated genes as well as wild-type one were cloned in pEX plasmids and expressed in E. coli BL21 (DE3) Tuner strain from Novagen (Madison, Wis., USA). The cell-free lysates were prepared from cells pregrown to OD₆₀₀=0.5 O.U. and induced by IPTG (1 mM ) within following 2 hrs. Pellets from 1.5 ml cultural medium were resuspended in 0.4 ml of 0.05 M KPi buffer (pH 5.1), containing 0.001 M EDTA, 0.15 M NaCl and 0.02% NaN₃, then sonicated for 1 min in 50% pulse regime with cooling using Ultrasonic processor XL (Misonix Inc., Farmingdale, N.Y.). Supernatant fractions, obtained after homogenates centrifugation for 15 minutes at 14,000 rpm, were denoted as cell-free extracts (CE) and used in PAI-1 activity assays. An aliquot of wild-type PAI-1 and each of mutant CEs, in serial dilutions from 1:40 up to 1:2560 in 0.05 M Tris HCl, pH 7.5 buffer, containing 0.15 M NaCl, 0.01% BSA, 0.0001% Tween 20, 0.02% SA, were analyzed for functional activity as measured by inhibitory activity against high molecular weight uPA using the single step chromogenic substrate assay as described (Lawrence et al. 1989 Eur. J. Biochem. 186: 529-539; Berkenpas et al., 1995). In addition, tPA inhibitory activity of mutant PAI-1 forms was evaluated by the direct formation of tPA:PAI-1 complex after 20 min of equimolar amounts tPA and PAI-1 incubation at 37° C. The reaction mixtures were analyzed by SDS-PAGE in 4-20% Tris-Glycine gradient gels under reducing conditions with prestained Sea Blue protein markers as a standards and followed by Coomassie Brilliant Blue-250 staining of the protein bands.

Binding Assays

Western Blot Assays

To evaluate the 33B8-binding- negative phenotype of selected PAI-1 mutants, an aliquots of CEs of E. coli BL21 (DE3)Tuner containing at least 5 ng of recombinant h PAI-1 were separated by SDS PAGE under reducing conditions; electro transferred to nitrocellulose membrane, blocked with 1% BSA/5% milk/TBS solution then probed with murine 33B8 mAbs and rabbit anti-h PAI-1 pAbs as the primary antibodies. The formed complexes were detected with HRP-conjugated goat anti-mouse or goat anti-rabbit immunoglobulins by ECL method according with the same procedure as for library screening (described above).

Surface Plasmon Resonance

The binding of purified h PAI-1 mutant proteins and E. coli BL21(DE3)Tuner CEs to specific murine anti-h PAI-1 33B8 mAbs has been also analyzed by surface plasmon resonance (SPR) method, using BIAcore™ 3000 optical Biosensor (BiacoreAB, Uppsala, Sweden). This method detects binding interactions in real time by measuring changes in the refractive index at a biospecific surface, enabling the association and dissociation rate constants to be calculated. Typically, 33B8 mAbs were coupled to a NHS/EDC-activated CM-5 research grade sensor chip flow cells, to yield approximately 1000 resonance units (RU) response. Individually, wild-type h PAI-1 in its active, latent, cleaved and complexed with uPA/tPA conformational forms as well as the single amino acid replacement and multiple mutant h PAI-1 proteins (0.001-1 μM ) in HBS-P buffer pH 7.4 were injected for 5 min at a flow rate of 20 μl/min followed by 10 min dissociation. Regeneration of the sensor chip surface was done with pulse of 20 μl 0.2 M Glycine buffer pH 1.5. All measurements were performed at 25° C. BIAcore 3000 Bioevaluation Software (Biacore AB, Uppsala, Sweden) program package was used for analysis of data and affinity constants calculations.

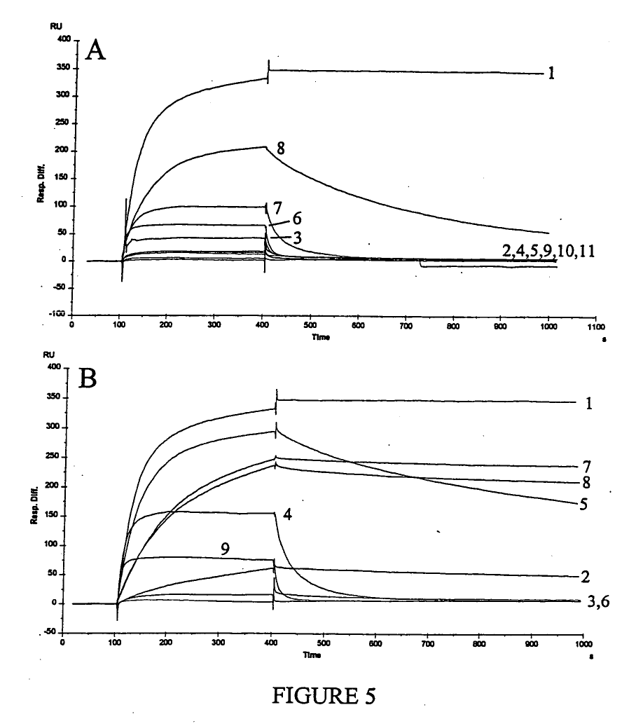

The identified mutants of PAI-1 that did not bind to the 33B8 antibody with high affinity and also were not inactivated by 33B8. This is shown in FIG. 5: antibody MA-33B8 (2.33 mN) was preincubated with purified wild type and mutant recombinant HPAI1 (1 mM at room temperature) after which the residual PAI-1 activity was measured at the intervals indicated. These data indicate that both wild-type PAI-1, and the previously identified mutant of PAI-1, 14-1b (Lawerence et al. 1989 Eur. J. Biochem. 186: 529-539; Berkenpas et al., 1995), are both rapidly inactivated by the antibody. In contrast, two of the newly constructed single point mutations that were identified in the screen and are shown in FIG. 2 (S331R and D89G) are not rapidly inactivated by 33B8. This demonstrates that mutations that disrupt binding of 33B8 to PAI-1 also prevent 33B8 for rapidly inactivating the PAI-1.

Method for Determining the Effect of Inhibition of PAI-1 Mutant Activity by Novel Chemical Entities

Novel chemical entities (NCE) are added at various concentrations to a buffer containing the PAI-1 mutant. Following mixing and incubation at room temperature, the target protease, either tissue plasminogen activator (tPA) or urokinase plasminogen activator (uPA), is added, and the combination of NCE, PAI-1 mutant and tPA/uPA is further incubated at room temperature. Following the second mixing and incubation, a chromogenic substrate for tPA/uPA is added and absorbance read in a spectrophometer at various times. Relative PAI-1 inhibitory activity is equal to the residual tPA/uPA activity in the NCE/mutant PAI-1 treatment compared to the activity in the presence of the mutant alone. By comparing these data to similar data generated by substituting wild-type PAI-1 for mutant PAI-1 in the presence of the NCE, the relative affinity of the NCE for the unique epitope within the mutant PAI-1 can be determined. Control treatments include the absence of any effect of the NCE on tPA/uPA activity alone. Only those compounds that have shown inhibitory activity against wild-type PAI-1 are further tested against mutant PAI-1. The inhibitory activity of the NCE against wild-type PAI-1 is determined by using a range of concentrations of the NCE, and estimating the molar concentration required for a 50% inhibitory effect using standard pharmacologic analysis. Under the conditions of the assay, those compounds ranked in the top 25% of all compounds tested are preferred, although compounds ranked from 26-75% may also have utility.

More specifically, Compound A, a NCE, is mixed with wild-type PAI-1 and produces a 100% reduction of the action of PAI-1 on uPA. Compound B, also a NCE, is mixed with wild-type PAI-1 and also produces a 100% reduction in the action of PAT-1 on uPA. Compound A is mixed with mutant PA-1 S331R which results in a 50% reduction in the action of this mutant on uPA. Compound B is mixed with mutant PAI-1 S331R resulting in a 0% reduction of the inhibitory action of the mutant PAI-1 on uPA. The complete absence of a reduction of the action of the Compound B on mutant PAI-1 S331R indicates that Compound B does not bind mutant PAI-1 S331 R, while the 50% reduction in the action of Compound A on mutant PAI-1 S331R indicates that Compound A binds mutant PAI-1 S331R. The binding site for Compound B is identified by the amino acid substitution in mutant PAI-1 S331R, which correspond to the binding epitope in MA33B8 for PAI-1.

This example could be expanded to include other mutations or binding sites. Compounds of interest for binding to the MA33B8 epitope in PAI-1 would either exhibit 50-100% reduction, 25-49% reduction, or 0-24% reduction in the action of mutants of PAI-1 on uPA. Those compounds with 0-24% reduction in the action of PAI-1 mutants on uPA would be considered as exhibiting activity.

This example includes but is not restricted to uPA as the target protease for PAI-1. By the same token, this example is not restricted in its significance to PAI-1 and mutants of PAI-1, as the metastable protein targeted for compound binding. Range in percent reduction in action of compounds on PAI-1 for determination of activity is given for the purpose of illustration, and may vary.

Compounds identified via the inventive screening methodology are inhibitors of PAI-1 and, hence, are useful in the treating, inhibiting, preventing or prophylaxis of processes, in a mammal, preferably a human, that involve the production and/or action of PAI-1. Thus, a compound of the present invention may be useful:
in the treatment or prevention of non-insulin dependent diabetes mellitus;
in the prevention of thrombotic events associated with coronary artery and cerebrovascular disease;
in the inhibiting of a disease process involving thrombotic and prothrombotic states, including but not limited to the formation of atherosclerotic plaques, venous and arterial thrombosis, myocardial ischemia, atrial fibrillation, deep vein thrombosis, coagulation syndromes, pulmonary fibrosis, cerebral thrombosis, thromboembolic complications of surgery, such as joint replacement, and peripheral arterial occlusion;
in the treatment of diseases associated with extracellular matrix accumulation, including but not limited to chronic obstructive pulmonary disease, polycystic ovary syndrome, restenosis, renovascular disease, and organ transplant rejection;
in the treatment of malignancies and diseases associated with neo-angiogenesis, such as diabetic retinopathy;
in conjunction with and following processes or procedures that maintain or that may affect blood vessel patency, including vascular surgery, vascular graft and stent patency, and organ, tissue and cell implantation and transplantation.
in the treatment of inflammatory diseases, septic shock and the vascular damage associated with infections;
in treatment of blood and blood products used in dialysis and blood storage in the fluid phase, especially ex vivo platelet aggregation;
during the analysis of blood chemistry in hospital settings, when the compound is added to human plasma, to determine the fibrinolytic capacity thereof;
in combination with prothrombolytic, fibrinolytic and anticoagulant agents;
in the treatment of cancers, including but not limited to breast and ovarian cancer, and, as an imaging agent, in the identification of metastatic cancers;
in the treatment of myelofibrosis, with myeloid metaplasia, by regulating stromal cell hyperplasia and effecting increases in extracellular matrix proteins;
in conjunction with protease inhibitor-containing, highly active anti-retroviral therapy (HAART), for the treatment of diseases, e.g., in HIV-1 infected patients receiving such therapy, that derive from fibrinolytic impairment and hyper-coagulability; and
in the treatment of diabetic nephropathy and renal dialysis associated with nephropathy.

More generally, the compounds of the present invention find use in improving coagulation homeostasis and in treating septicemia, obesity, insulin resistance, psoriasis and other proliferative diseases, cerebrovascular diseases, microvascular disease, hypertension, dementia, osteoporosis, arthritis, asthma, heart failure, arrhythmia, and angina. An inventive compound also can be employed as a hormone replacement agent, for example, in treating, preventing, or reversing progression of atherosclerosis, osteoporosis, and osteopenia. It also can be employed for reducing inflammatory markers, reducing C-reactive protein, and preventing or treating low grade vascular inflammation, stroke, dementia and coronary heart disease, as well as in primary and secondary prevention of myocardial infarction, stable and unstable angina, in primary prevention and secondary prevention of cardiovascular events, in ameliorating peripheral vascular disease, peripheral arterial disease, acute vascular syndromes, in reducing the risk of undergoing a myocardial revascularization procedure, in treating microvascular diseases, such as nephropathy, neuropathy, retinopathy and nephrotic syndrome, hypertension, Type I and 2 diabetes and related diseases, hyperglycemia, hyperinsulinemia, malignant lesions, premalignant lesions, gastrointestinal malignancies, liposarcomas and epithelial tumors, and all forms of cerebrovascular diseases, and in improving endothelial function.

The compounds of the present invention also are useful in the treatment of Alzheimer's disease. This approach can be characterized, in accordance with the invention, as a method for increasing or normalizing plasmin levels in a subject, particularly one who is experiencing or who is subject to risk for Alzheimer's disease.

All of the publications and patent documents cited herein are incorporated in their entirety by reference.

We claim:

1. A mutant proteinase inhibitor comprising a wild-type proteinase inhibitor amino acid sequence with at least one mutation in at least one epitope of said amino acid sequence, wherein said mutation alters the binding of said mutant proteinase inhibitor to an anti-proteinase inhibitor antibody as compared to the binding of said wild-type proteinase inhibitor to said anti-proteinase inhibitor antibody.

2. The mutant proteinase inhibitor of claim 1, wherein said anti-proteinase inhibitor antibody has a lower binding affinity to said mutated epitope of said mutant proteinase inhibitor than said anti-proteinase inhibitor antibody has for said wild-type proteinase inhibitor.

3. The mutant proteinase inhibitor of claim 1, wherein said anti-proteinase inhibitor antibody affects a measurable activity of said wild-type proteinase inhibitor.

4. The mutant proteinase inhibitor of claim 3, wherein said measurable activity is the inhibitory activity of said wild-type proteinase inhibitor against a target proteinase.

5. The mutant proteinase inhibitor of claim 1, wherein said anti-proteinase inhibitor antibody does not bind to said mutated epitope of said mutated proteinase inhibitor.

6. The mutant proteinase inhibitor according to claim 1, wherein said mutant proteinase inhibitor is a mutant of wild-type plasminogen activator inhibitor (PAI).

7. The mutant proteinase inhibitor according to claim 6, wherein said mutant PAI is a mutant of wild-type PAI-1.

8. The mutant proteinase inhibitor according to claim 7, wherein the amino acid sequence of said wild-type PAI-1 is depicted in FIG. 1.

9. The mutant proteinase inhibitor according to claim 8, wherein said anti-proteinase inhibitor antibody is an anti-PAI monoclonal antibody.

10. The mutant proteinase inhibitor according to claim 9, wherein said anti-PAI monoclonal antibody is MA-33B8 or 31 C9.

11. The mutant proteinase inhibitor according to claim 8, wherein said amino acid sequence comprises a substitution at one or more amino acid residues selected from the group consisting of 1, 2, 13, 82, 87, 88, 89, 92, 95, 110, 115, 127, 151, 174, 196, 202, 212, 218, 223, 230, 241, 283, 300, 305, 308, 329, 331, 323, 339, 351 and 354.

12. The mutant proteinase inhibitor according to claim 11, wherein said mutant proteinase inhibitor comprises a substitution at one or more amino acid residues selected from the group consisting of 87, 88, 89, 174, 230, 232, 329 and 331.

13. The mutant proteinase inhibitor according to claim 10, wherein said mutant proteinase inhibitor comprises a substitution at amino acid residues selected from the group consisting of 87, 88, 89, 174, 230, 232, 329 and 331.

14. The mutant proteinase inhibitor according to claim 12, wherein said mutant comprises at least one amino acid substitution at amino acid 87.

15. The mutant proteinase inhibitor according to claim 14, wherein said amino acid substitution at amino acid 87 is changing asparagine to aspartic acid.

16. The mutant proteinase inhibitor according to claim 12, wherein said mutant comprises at least one amino substitution at amino acid 230.

17. The mutant proteinase inhibitor according to claim 16, wherein said amino substitution at amino acid 230 is changing glycine to valine.

18. The mutant proteinase inhibitor according to claim 12, wherein said mutant comprises a substitution at least one amino acid substitution at amino acid 331.

19. The mutant proteinase inhibitor according to claim 18, wherein said amino acid substitution at amino acid 331 is changing serine to arginine.

20. A fragment of the mutant proteinase inhibitor of claim 1, wherein said fragment comprises at least one mutation in at least one epitope of said amino acid sequence.

21. A nucleic acid sequence comprising a nucleotide sequence encoding said mutant proteinase inhibitor according to claim 1 or a fragment of said mutant proteinase inhibitor, wherein said mutant proteinase inhibitor or said fragment comprises at least one mutation in at least one epitope of said amino acid sequence.

22. A nucleic acid construct comprising the nucleic acid sequence according to claim 21 operably linked to regulatory control sequences that effect the expression of said nucleic acid sequence.

23. A vector comprising the nucleic acid construct according to claim 22.

24. A host transformed or transfected with a nucleic acid construct according to claim 22.

25. A method for producing a mutant proteinase inhibitor or a fragment thereof comprising culturing the host according to claim 24 under conditions wherein said nucleic acid sequence is expressed.

26. A method of screening at least one compound that affects the activity of a proteinase inhibitor comprising:
(a) incubating a compound with:
(i) a mutant proteinase inhibitor of claim 1 or a fragment thereof, wherein said inhibitor or said fragment comprises at least one mutation in at least one epitope of said amino acid sequence; and separately incubating said compound with:
(ii) a wild-type proteinase inhibitor or a fragment thereof;
(b) measuring the binding of said mutant proteinase inhibitor or said fragment and said wild-type proteinase inhibitor or said fragment with said compound; and
(c) comparing the binding of said compound to said mutant proteinase inhibitor or said fragment with the binding of said compound to a wild-type proteinase inhibitor or said fragment.

27. The method of claim 26, further comprising:
(d) selecting a compound that binds less strongly with said mutant proteinase inhibitor or said fragment as compared to the binding of said compound to said wild-type proteinase inhibitor or said fragment.

28. The method of claim 27, wherein said compound binds to said mutant proteinase inhibitor or said fragment about 95% or less than the binding of said compound to said wild-type proteinase inhibitor or fragment.

29. The method of claim 27, wherein said compound binds to said mutant proteinase inhibitor or said fragment about 75% or less than the binding of said compound to said wild-type proteinase inhibitor or fragment thereof.

30. The method of claim 27, wherein said compound binds to said mutant proteinase inhibitor or said fragment about 50% or less than the binding of said compound to said wild-type proteinase inhibitor or fragment thereof.

31. The method of claim 27, wherein said compound binds to said mutant proteinase inhibitor or said fragment about 24% or less than the binding of said compound to said wild-type proteinase inhibitor or fragment thereof.

32. The method of claim 27, wherein said compound does not bind to said mutant proteinase inhibitor or said fragment but does bind to said wild-type proteinase inhibitor or said fragment.

33. A method of screening at least one compound that affects the inhibitory activity of a proteinase inhibitor comprising:
(a) incubating a compound with:
(i) a mutant proteinase inhibitor of claim 1 or a fragment thereof, wherein said inhibitor or said fragment comprises at least one mutation in at least one epitope of said amino acid sequence, and separately incubating said compound with:
(ii) a wild-type proteinase inhibitor or a fragment thereof;
(b) measuring the effect of said compound in (a) on the inhibitory activity of said mutant proteinase inhibitor or said fragment and said wild-type proteinase inhibitior on a target proteinase; and
(c) comparing the effect of said compound in (b) on the inhibitory activity of said mutant proteinase inhibitor or said fragment with the effect of said compound on the inhibitory activity of a wild-type proteinase inhibitor or said fragment.

34. The method of claim 33, further comprising:
(d) when said compound affects the inhibitory activity of said wild-type proteinase, selecting a compound that has less of an effect on the inhibitory activity of said mutant proteinase inhibitor or said fragment in (b) than the effect on the inhibitory activity of said wild-type proteinase inhibitor or said fragment.

35. The method of claim 34, wherein said mutant proteinase inhibitor or fragment thereof retains from about 5-100% of its inhibitory activity against the target proteinase as measured in the absence of the compound.

36. The method of claim 34, wherein said mutant proteinase inhibitor or fragment thereof retains at least about 25% of its inhibitory activity against the target proteinase as measured in the absence of the compound.

37. The method of claim 34, wherein said mutant proteinase inhibitor or fragment thereof retains at least about 50% of its inhibitory activity against the target proteinase as measured in the absence of the compound.

38. The method of claim 34, wherein said mutant proteinase inhibitor or fragment thereof retains at least about 75% of its inhibitory activity against the target proteinase as measured in the absence of the compound.

39. The method of claim 34, wherein said compound has substantially no effect on the inhibitory activity of said mutant proteinase inhibitor or said fragment.

40. The method of claim 34, wherein said measuring of step (b) comprises adding a target proteinase to said mutant proteinase inhibitor and said compound of (a) to form a first mixture and to said wild-type proteinase inhibitor of (a) to form a second mixture and incubating said first and second mixtures.

41. The method of claim 34, wherein said measuring of (b) comprises measuring said target proteinase activity.

42. The method of claim 41, wherein said measuring of said target proteinase activity comprises adding a substrate of said target proteinase and measuring the enzymatic conversion of said substrate.

43. The method of claim 34, wherein said screening further comprises control treatments comprising:
(e) performing a parallel set of steps (a) through (d), except that no compound is added in step (a).

44. The method of claim 34, wherein said method further comprises performing a control incubation, wherein said target proteinase activity is measured without the addition of said mutant proteinase inhibitor or said fragment and ef said wild-type proteinase inhibitor or said fragment in (a).

45. The method of claim 40, wherein said method further comprises performing a control incubation, wherein said target proteinase activity is measured without the addition of said mutant proteinase inhibitor or said fragment, said wild-type proteinase inhibitor or said fragment and said compound in (a).

46. A method of mapping at least one compound binding site in a metastable protein comprising:
(a) incubating a compound with:
(i) a mutant metastable protein or a fragment thereof, and separately incubating said compound with:
(ii) a wild-type metastable protein or a fragment thereof;
(b) measuring the effect of said compound in (a) on a measurable activity of said mutant metastable protein or said fragment and said wild-type metastable protein or said fragment; and
(c) comparing the effect of said compound in (b) on the measurable activity of said mutant metastable protein or said fragment with the effect of said compound on the measurable activity of said wild-type metastable protein or said fragment.

47. The method of claim 46, further comprising:
(d) when said compound affects the measurable activity of said wild-type stable protein, selecting a compound that has less of an effect on the activity of said mutant metastable protein or said fragment in (b) than the effect on the activity of said wild-type stable protein or said fragment.

48. The method of claim 47, wherein said mutant metastable protein or fragment thereof retains from about 5-100% of its measurable activity in the presence of the compound.

49. The method of claim 47, wherein said mutant metastable protein or fragment thereof retains at least about 25% of its measurable activity in the presence of the compound.

50. The method of claim 47, wherein said mutant metastable protein or fragment thereof retains at least about 50% of its measurable activity in the presence of the compound.

51. The method of claim 47, wherein said mutant metastable protein or fragment thereof retains at least about 75% of its measurable activity in the presence of the compound.

52. The method of claim 47, wherein said compound has substantially no effect on the inhibitory activity of said mutant metastable protein or fragment thereof.

53. The method of claim 47, wherein said screening further comprises control treatments comprising:
(e) performing a parallel set of steps (a) through (d), except that no compound is added in step (a).

54. The method of claim 47, wherein said method further comprises performing a control incubation, wherein said measurable activity is measured without the addition of said mutant metastable protein or said fragment and said wild-type metastable protein or said fragment in (a).

55. The method of claim 47, wherein said method further comprises performing a control incubation, wherein said measurable activity is measured without the addition of said mutant metastable protein or said fragment, said wild-type metastable protein or said fragment and said compound in (a).

56. The method of claim 47, wherein said mutant metastable protein or said fragment is prepared by introducing at least one mutation in at least one epitope of the amino acid sequence of said wild-type metastable protein or said fragment, wherein said mutation alters the binding of said mutant metastable protein or fragment thereof to an antibody that binds to said wild-type metastable protein or fragment thereof.


SEQUENCE LISTING (12 sequences)

SEQ ID NO: 1 - DNA, 2876 bp, Homo sapiens
  CDS (76)..(1281)
gaattcctgcagctcagcagccgccgccagagcaggacgaaccgccaatcgcaaggcacctctgagaacttcaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagc

SEQ ID NO: 2 - protein, 402 aa, Homo sapiens [sequence omitted]

SEQ ID NO: 3 - DNA, 33 bp, Artificial Sequence
  Description of Artificial Sequence Mutagenic primer
ggagctcatggggccatgggacaaggatgagat

SEQ ID NO: 4 - DNA, 33 bp, Artificial Sequence
  Description of Artificial Sequence Mutagenic primer
gctcatggggccatggaacgaggatgagatcag

SEQ ID NO: 5 - DNA, 23 bp, Artificial Sequence
  Description of Artificial Sequence Mutagenic primer
catggaacaagggtgagatcagc

SEQ ID NO: 6 - DNA, 39 bp, Artificial Sequence
  Description of Artificial Sequence Mutagenic primer
gccctctacttcaacggccggtggaagactcccttcccc

SEQ ID NO: 7 - DNA, 38 bp, Artificial Sequence
  Description of Artificial Sequence Mutagenic primer
ctgaactgccctaccacgtggacaccctcagcatgttc

SEQ ID NO: 8 - DNA, 22 bp, Artificial Sequence
  Description of Artificial Sequence Mutagenic primer
ccacggggactccctcagcatg

SEQ ID NO: 9 - DNA, 33 bp, Artificial Sequence
  Description of Artificial Sequence Mutagenic primer
gtgaagatcgaggtgatcgagagtggcacggtg

SEQ ID NO: 10 - DNA, 21 bp, Artificial Sequence
  Description of Artificial Sequence Mutagenic primer
gtgaacgagagaggcacggtg

SEQ ID NO: 11 - protein, 5 aa, Artificial Sequence
  Description of Artificial Sequence Synthetic peptide
SAVHH

SEQ ID NO: 12 - protein, 5 aa, Artificial Sequence
  Description of Artificial Sequence Synthetic peptide
VHHPP